(19) United States
(12) Patent Application Publication
(10) Pub. No.: US 2008/0152894 A1
(43) Pub. Date: Jun. 26, 2008

(54) Treated substrates having improved delivery of impregnated ingredients

(75) Inventors: Thomas W. Beihoffer, Arlington Heights, IL (US); Kevin Cureton, Evanston, IL (US)

Correspondence Address: MARSHALL, GERSTEIN & BORUN LLP, 233 S. WACKER DRIVE, SUITE 6300, SEARS TOWER, CHICAGO, IL, 60606, US

(73) Assignee: AMCOL HEALTH & BEAUTY SOLUTIONS, Arlington Heights, IL (US)

(21) Appl. No.: 11/643,327

(22) Filed: Dec. 20, 2006

(51) Int. Cl. A61K 8/02; A01N 25/34; A61K 9/70; A61Q 17/04; A61Q 19/02; C11D 17/04

(52) U.S. Cl. 428/317.9; 428/304.4; 424/414; 424/443; 424/59; 424/62; 510/438

(57) ABSTRACT

An article of manufacture containing polymeric microparticles, optionally loaded with an active agent, impregnated into a porous substrate is disclosed.

### FIELD OF THE INVENTION

The present invention relates to a substrate having polymeric microparticles impregnated therein. More particularly, the present invention relates to articles of manufacture, such as a wipe product, comprising a particle-impregnated substrate that efficiently transfers the particles from the substrate to a surface contacted with the impregnated substrate. The polymeric microparticles are transferred from the substrate to a contacted surface to impart a benefit to an animate or inanimate surface. The benefit can be attributed to the polymeric microparticles themselves, to an active agent incorporated into the microparticles, or both.

### BACKGROUND OF THE INVENTION

The use of fabrics, sponges, and other porous or intersticed substrates to retain an active agent is known The products are commonly termed “wipes.” The active agents can range widely in identity and function, and can serve a wide range of applications, including therapeutic, cosmetic, hygienic, and preventive. Wipes are utilized in personal care products, cosmetics, toiletries, fragrances, pharmaceutical products, and household and industrial products.

Wipes are convenient products, used by simply wiping a surface with an impregnated substrate. In use, impregnated ingredients are transferred from the substrate to the wiped surface to perform their intended function.

The explosive growth of wipe products has been well documented by market research organizations and periodicals. Once a market dominated by the simple baby wipe, it has grown into an industry providing a wide variety of wipe products, including wet, dry, and chemically treated dry wipes. Regardless of the market, the majority of present-day wipe products perform a cleansing function as a primary benefit, with convenience being the driver for their use.

However, convenience alone will not continue to drive the double-digit growth of wipe products. In order for the wipe product market to continue to grow, new technology is needed to enable greater application diversity, i.e., expanding the benefits of wipe products beyond convenience and cleansing into treatment and protection. For this to occur, wipe products must deliver benefits beyond those a consumer can gain from a bottle or tube of a lotion, cream, or gel.

Wipe products for delivery of an active agent to a surface are known in the art. For example, U.S. Pat. No. 5,156,843 discloses a particle delivery system as a carrier for active agents. The particles are dispersed within porous fabric materials. The active agents are released from the particles onto the fabric, and the active agents then are transferred from the fabric to a surface wiped with the surface.

U.S. Pat. No. 4,904,524 discloses a porous sheet impregnated with an aqueous lotion comprising a hydrophobic active agent entrapped in polymeric beads, wherein the impregnated particles are concentrated near the surface of the porous sheet by dusting or spraying dry particles on the surface of a porous substrate.

Microparticle delivery systems also have been disclosed. These delivery systems typically comprise a polymeric microparticle that is capable of absorbing or adsorbing a hydrophilic active agent, a hydrophobic active agent, or both. Typically, the polymeric microparticles are capable of absorbing or adsorbing more than their weight of an active agent.

However, even in view of the substantial art relating to wipe products and polymeric microparticles, the art does not address the relative properties of the wipe substrate and the polymeric microparticles that facilitate the transfer of the microparticles from the wipe product to a treated surface. The present invention addresses these relative properties in order to provide a particle-impregnated substrate having an improved ability to deliver polymeric microparticles, and any active agent loaded therein, from the substrate to a treated surface.

### SUMMARY OF THE INVENTION

In accordance with the present invention, it has been discovered that the transfer of an active agent and/or a polymeric microparticle delivery system to target surfaces from substrates can be improved by control of properties of the particulate delivery systems and the substrate.

Surprisingly, when the substrate has a proper porosity, as measured by Frazier number, in combination with a polymeric microparticle delivery system having a proper particle size, transfer of an active ingredient and/or the microparticles themselves from the substrate to a target surface is greatly improved.

The present invention is directed to articles of manufacture comprising a porous substrate having polymeric microparticles impregnated therein. The article of manufacture can be a wipe product, a garment, a bed sheet, a pillow case, a carpet, gloves, an applicator, a diaper, a cloth towel, a paper sheet, or a paper towel, for example. For brevity, the present specification is directed particularly to wipe products, but it should be understood that this is for convenience only and is not intended to be limiting.

The polymeric microparticles can have an active agent loaded thereon. The resulting article is used to deliver the polymeric microparticles, and any active agent loaded thereon, to an animate or inanimate surface contacted with the wipe.

One aspect of the present invention is to provide an article of manufacture comprising a microparticle-impregnated substrate having an improved ability to transfer the microparticles from the substrate to a contacted surface. This improved transfer of the microparticles from the substrate to a surface is attributed to (a) the particle size distribution of the polymeric microparticles and (b) the Frazier number of the substrate, which, when judiciously selected, improve transfer of the microparticles from the substrate to a contacted, such as a wiped, surface.

Therefore, one embodiment of the present invention is to provide an article of manufacture wherein the substrate has a Frazier number less than about 210.

Another embodiment of the present invention is to provide an article of manufacture wherein the substrate is impregnated with polymeric microparticles having a mean particle size diameter of greater than one micron.

Still another embodiment of the present invention is to provide an article of manufacture comprising a substrate having a Frazier number less than 210 and having polymeric microparticles of median particle size diameter greater than one micron impregnated therein.

In one embodiment, the microparticles are present near the surface of a substrate. In another embodiment, the microparticles are distributed throughout the volume of the substrate.

In still another embodiment, an active agent is loaded onto the microparticles prior to impregnating the substrate of the article with the microparticles.

In yet another embodiment, the polymeric microparticles are impregnated into a substrate to provide an article suitable for application to the skin of a mammal. The article can further contain ingredients in addition to the microparticles.

Another embodiment of the present invention is to provide a method of treating a surface, for example skin or a hard surface, by contacting the surface with an article comprising a substrate having polymeric microparticles impregnated therein to provide a surface-beneficial property.

Still another embodiment of the present invention is to provide an article of manufacture, such as a wipe product, a garment, a cloth, a glove, a bed sheet, a sheet of a diaper, an applicator, a cloth towel, a linen, a napkin, a paper towel or sheet, or similar sheet-like articles comprising a substrate having a Frazier number less than 160 and polymeric microparticles of median particle size greater than one micron impregnated therein.

Wipe products of the present invention are useful as skin lotions, creams, body rinses, skin-cleansing agents, skin-coloring compositions, foundation liquids and powders (compressed or loose), topical medicaments, skin-treatment products, hard surface cleansers, hard surface disinfectants, and similar personal care, industrial, and institutional products.

These and other aspects and novel features of the present invention will become apparent from the following detailed description of the preferred embodiments.

### DETAILED DESCRIPTION OF THE PREFERRED EMBODIMENTS

The present invention is directed to an article of manufacture, such as a wipe product comprising a porous substrate having polymeric microparticles impregnated therein. The polymeric microparticles optionally have an active agent incorporated therein. The article demonstrates an improved ability to transfer the polymeric microparticles from the substrate to a contacted, e.g., wiped, surface because of the Frazier number of the substrate and the median particle size diameter of the microparticles.

A substrate of the article generally is an intersticed material, such as a cloth or fabric-like sheet, comprising fibers or fiber blends designed to impart desired strength and wetting properties to the substrate. A variety of substrate types and constructions are envisioned as suitable for the invention. Nonlimiting examples of suitable substrates include woven and nonwoven webs, such as spun-lace, melt-blown, and air-laid fabrics.

Cellulosic fibrous webs are preferred as the porous substrate for an article of the present invention because of a low cost and biodegradability. Other preferred porous substrates are paper, air-laid, and carded nonwoven webs. Spun-bonded and spun-lace webs are also suitable as the porous substrate. For applications where cost and/or biodegradability are not an issue, alveolar polymeric films, foam, and other porous substrates may be employed.

Nonfibrous substrates, such as foams or perforated films, also are envisioned as suitable substrates for the article. The substrate can be in the form of a sheet, pad, or applicator, for example. The substrate can be laminated with other materials, such as other fabrics or films, to achieve a desired form for a microparticle application. The substrate can be wetted with aqueous or nonaqueous liquids, or can be dry, prior to loading of the polymeric microparticles.

In accordance with an important feature of the present invention, the substrate, independent of the material of construction, has a Frazier number of less than 210, preferably less than about 200, about 190, about 185, about 180, about 175, about 170, about 165, or about 160, and more preferably less than about 150, about 125, about 100, or about 75. The Frazier number of the substrate can be as low as about 30.

The Frazier number is a measure of the permeability of a substrate, measured by the flow of air through a substrate under a set of defined conditions. The Frazier Differential Pressure Air Permeability Instrument, from the Frazier Precision Instrument Company, Inc., Hagerstown, Md. 21740, is the standard in the industry for measuring air permeability. The term “Frazier Number” (or CFM) has been developed over the years in regard to permeability or porosity. Frazier numbers are provided by substrate manufacturers and are reported in units of liters/minute/100 cm² at 12.7 mm (i.e., 0.5 inches) of water differential air pressure.

As used herein, a “target surface” is a surface contacted by a present article to deliver a desired benefit. The benefit can include delivery of active ingredients loaded onto the polymeric microparticles, the delivery of the polymeric microparticles per se, or both. Nonlimiting examples of target surfaces include skin, teeth, and hair for cosmetic and drug applications, and hard surfaces, such as countertops, for cleaning applications.

As used herein, the term “substrate working layer” is the surface of the substrate that is applied to, e.g., contacts, a target surface with an intent to deliver a benefit to the target surface. For a multilayer substrate, e.g., one produced by laminating several layers of material together, the working layer is the outermost, or external, substrate layer that contacts the target surface.

Polymeric microparticles are a delivery system often used in personal care and pharmaceutical formulations to extend release of an active ingredient, to protect the active ingredient from decomposition in a composition, and/or to enable formulation of the active ingredient into a composition due to difficulties, such as solubility or formulation esthetics.

Polymeric microparticle delivery systems comprise discrete, free-flowing particles which can absorb, adsorb, entrap, or otherwise retain an active agent in a polymeric matrix. Such microparticles can provide a controlled release of the active agent over time either by rupture of the microparticle, whereby the active agent is released when sufficient pressure or shearing action is applied to the microparticle, or the microparticle may be semipermeable or porous which allows the active agent to diffuse from the particle. In some embodiments, the polymeric microparticles themselves, without a loaded active agent, provide a desired benefit, i.e., an oil absorption function. Additionally, the microparticle delivery system can deliver multiple active agents in addition to itself.

The term “polymeric microparticle delivery system” encompasses microparticles and microcapsules generally, which are a well-known form of polymeric beads formed by emulsion polymerization, precipitation polymerization, and other methods. Absorbent polymeric microparticles useful in the present invention have an ability to absorb several times their weight of a liquid compound, such as a skin care compound.

One preferred class of adsorbent microparticles is prepared by a suspension polymerization technique, as set forth in U.S. Pat. Nos. 5,677,407; 5,712,358; 5,777,054; 5,830,967; 5,834,577, 5,955,552; and 6,107,429, each incorporated herein by reference (available commercially under the tradename of POLY-PORE® E200, INCI name, allylmethacrylate copolymer, from AMCOL International, Arlington Heights, Ill.). Another preferred class of adsorbent microparticles is prepared by a precipitation polymerization technique, as set forth in U.S. Pat. Nos. 5,830,960; 5,837,790, 6,248,849; and 6,387,995, each incorporated herein by reference (sold under the tradename of POLY-PORE® L200 by AMCOL International, Arlington Heights, Ill.). These adsorbent microparticles also can be modified after loading incorporation of an active agent to modify the rate of release of such an agent, as set forth in U.S. Pat. No. 6,491,953, incorporated herein by reference.

Another useful class of adsorbent polymers prepared by a precipitation polymerization technique is disclosed in U.S. Pat. Nos. 4,962,170; 4,948,818; and 4,962,133, each incorporated herein by reference, and are commercially available under the tradename POLYTRAP from AMCOL International. Other useful, commercially available adsorbent polymers include, for example, MICROSPONGE (a copolymer of methyl methacrylate and ethylene glycol dimethacrylate), available from Cardinal Health, Sommerset, N.J., and Poly-HIPE polymers (e.g., a copolymer of 2-ethylhexyl acrylate, styrene, and divinylbenzene) available from Biopore Corporation, Mountain View, Calif.

In particular, the adsorbent polymer microparticles prepared by the suspension polymerization technique, e.g., POLY-PORE® E200, are a highly porous and highly crosslinked polymer in the form of open (i.e., broken) spheres and sphere sections characterized by a median particle size diameter of about 0.5 micron to about 3,000 microns, preferably greater than 1 to about 300 microns, more preferably greater than 1 to about 100 microns, and most preferably about 1 to about 80 microns. A significant portion of the spheres is about 20 microns in diameter.

It should be understood that prior to impregnating the substrate with the polymeric microparticles, the polymeric microparticles can have a median particle size diameter of one micron or less. However, after loading an active agent onto the microparticles and/or after impregnating the substrate, the polymeric microparticles have a median particle size diameter of greater than one micron.

The polymeric microparticles are oil and water adsorbent, and have an extremely low bulk density of about 0.008 gm/cc to about 0.1 gm/cc, preferably about 0.009 gm/cc to about 0.07 gm/cc, and more preferably about 0.0095 gm/cc to about 0.04-0.05 gm/cc. The microparticles are capable of holding and releasing oleophilic (i.e., oil soluble or dispersible), as well as hydrophilic (i.e., water soluble or dispersible), active agents, individually, or both oleophilic and hydrophilic compounds simultaneously. The microparticles also are capable of adsorbing and absorbing oleophilic and hydrophilic components from a surface, and therefore can provide a benefit even when an active agent is not loaded onto the microparticles.

Adsorbent polymer microparticles prepared by the suspension polymerization technique are prepared from at least two polyunsaturated monomers, preferably allyl methacrylate and an ethylene glycol dimethacrylate, and, optionally, monounsaturated monomers. The microparticles are characterized by being open to their interior, due either to particle fracture upon removal of a porogen after polymerization or to subsequent milling. The microparticles typically have a median particle size diameter of less than about 50 microns, preferably less than about 25 microns, and have a total adsorption capacity for organic liquids, e.g., mineral oil, that is at least about 72% by weight, preferably at least about 93% by weight, and an adsorption capacity for hydrophilic compounds and aqueous solutions of about 70% to about 89% by weight, preferably about 75% to about 89% by weight, calculated as weight of material adsorbed divided by total weight of material adsorbed plus dry weight of polymer. In a preferred embodiment, the broken sphere microparticles are characterized by a median particle size diameter of greater than 1 to about 50 microns, more preferably of greater than 1 to about 25 microns, most preferably, of greater than 1 to about 20 microns, after impregnation into the substrate.

Preferred polymeric microparticle delivery systems comprise a copolymer of allyl methacrylate and ethylene glycol dimethacrylate, a copolymer of ethylene glycol dimethacrylate and lauryl methacrylate, a copolymer of methyl methacrylate and ethylene glycol dimethacrylate, a copolymer of 2-ethylhexyl acrylate, styrene, and divinylbenzene, and mixtures thereof.

Specific polymeric microparticles useful in the present invention can be the previously described POLY-PORE® E200, POLY-PORE® L200, POLYTRAP, MICROSPONGE, or Poly-HIPE particles, for example. An active agent can be loaded onto such microparticles to provide microparticles containing 0 to about 80 wt. %, preferably about 0.01% to about 70 wt. %, and most preferably about 1% to about 50 wt. %, by weight of the loaded microparticles. The loaded microparticles typically are incorporated into a substrate in an amount to provide about 0.5% to about 40%, preferably about 2% to about 30%, and typically about 1% to about 20%, by weight, of polymeric microparticles in the wipe product.

In accordance with the present invention, when an active agent is loaded onto the polymeric microparticles, an active agent first is loaded onto the microparticles. Loading of the active agent onto the microparticles also is referred to herein as an “entrapment.” As used herein, the term “loaded microparticle” refers to a microparticle having an ingredient added thereto. Loading of an ingredient includes one or more of impregnating, imbedding, entrapping, absorbing, and adsorbing of the active agent and other ingredients into or onto the polymeric microparticles.

After loading an active agent on the microparticles, the microparticles are impregnated into the substrate. The microparticles first can be dispersed in an oleophilic or hydrophilic liquid to facilitate impregnation of the particles into the substrate. In other embodiments, the substrate first is wetted with an oleophilic or hydrophilic liquid, then the microparticles are impregnated into or applied onto the substrate. In still another embodiment, dry polymeric microparticles are added to a dry, i.e., unwetted, substrate.

The polymeric microparticle delivery systems can be applied to the substrates via any suitable method. Nonlimiting examples include direct application of the polymeric particle delivery system to the substrate by sprinkling, dusting, or spraying, for example. Preferred methods involve dispersing the microparticles in an aqueous or nonaqueous liquid or mixture of liquids, then applying the dispersion onto the substrate. The dispersion can be sprayed, coated, dipped, infused, or otherwise applied to a substrate.

In still another embodiment, prior to introducing the polymeric microparticles to the substrate, a barrier layer (i.e., a secondary entrapment), optionally, can be applied to the loaded microparticles to prevent rapid diffusion of an active agent from the microparticles, and to protect the active agent from the surrounding environment until application. This is especially effective for reactive compounds, like benzoyl peroxide, retinol, or a retinoid. Also, the melting point of the barrier layer can be selected such that the barrier layer melts at a higher temperature than the highest temperature that the polymeric microparticles will be exposed either during storage or during accelerated aging of the wipe product.

Examples of materials that can be used as a barrier layer, also termed a secondary loading or secondary entrapment, include, but are not limited to, low melting alcohols (C₈ through C₂₀) and fatty alcohols ethoxylated with one to three moles of ethylene oxide. Examples of fatty alcohols and alkoxylated fatty alcohols include, but are not limited to, behenyl alcohol, caprylic alcohol, cetyl alcohol, cetaryl alcohol, decyl alcohol, lauryl alcohol, isocetyl alcohol, myristyl alcohol, oleyl alcohol, stearyl alcohol, tallow alcohol, steareth-2, ceteth-1, cetearth-3, and laureth-2. Additional fatty alcohols and alkoxylated alcohols are listed in the International Cosmetic Ingredient Dictionary and Handbook, Tenth Edition, Volume 3, pages 2127 and pages 2067-2073 (2004), incorporated herein by reference.

Another class of materials that can be used a barrier layer is the C₈ to C₁₂ fatty acids, including, but not limited to, stearic acid, capric acid, behenic acid, caprylic acid, lauric acid, myristic acid, tallow acid, oleic acid, palmitic acid, isostearic acid and additional fatty acids listed in the International Cosmetic Ingredient Dictionary and Handbook, Tenth Edition, Volume 3, page 2126-2127 (2004), incorporated herein by reference. The barrier material also can be a hydrocarbon, like mineral oil, 1-decene dimer, polydecene, paraffin, petrolatum, vegetable-derived petrolatum or isoparafin. Another class of barrier materials is waxes, like mink wax, carnauba wax, and candelilla wax, for example, and synthetic waxes, like silicone waxes, polyethylene, and polypropylene, for example.

Fats and oils can be useful barrier material agents, which include, for example, but are not limited to, lanolin oil, linseed oil, coconut oil, olive oil, menhaden oil, castor oil, soybean oil, tall oil, rapeseed oil, palm oil, and neatsfoot oil, and additional fats and oils listed in the International Cosmetic Ingredient Dictionary and Handbook, Tenth Edition, Volume 3 (2004), pages 2124-2126. Other useful classes of barrier materials include a water-insoluble ester having at least 10 carbon atoms, and preferable 10 to about 32 carbon atoms. Numerous esters are listed in International Cosmetic Ingredient Dictionary and Handbook, Tenth Edition, pages 2115-2123 (2004).

Alternatively, an active agent can be mixed with a barrier layer material, then loaded onto the polymeric microparticles. In the case of liquid active agents, the materials disclosed above as barrier materials also can be used as an additive for thickening the liquid active agent, and thereby minimize premature diffusion of the active agent from the polymeric microparticles.

When present, the barrier layer can be about 5% to about 70%, by total weight of the loaded polymeric microparticles. In a preferred embodiment, the barrier layer is present at about 10% to about 50 wt. %, by total weight of the loaded polymeric microparticles.

An article of the present invention therefore comprises a substrate containing polymeric microparticles, optionally loaded with an active agent and an optional barrier material. The article also can contain other ingredients well known in the art, e.g., an alcohol, water, dye, fragrance, or similar ingredients.

In accordance with an important feature of the present invention, the polymeric microparticles of the article have a median particle size diameter of greater than one micron, for example, greater than one micron to about 3000 microns. Polymeric microparticles having a median particle size diameter of greater than one micron, incorporated into a substrate having a Frazier number of less than 160, provide an article, e.g., a wipe product, having an improved ability to deliver the polymeric microparticles to a contacted surface.

A variety of active agents can be incorporated into the polymeric microparticles such that a present article can impart a benefit to a contacted surface, either animate or inanimate. In some embodiments, the polymeric microparticles themselves provide a benefit, e.g., oil absorption. The active agent can be any hydrophobic or hydrophilic compound that is loaded onto the polymeric microparticles with the intent of delivering the active agent from the article and the polymeric microparticles to a target surface.

As previously stated, the polymeric microparticles can function as active agents. Active agents that can be loaded onto the polymeric microparticles include, but are not limited to: hormones, analgesics, anesthetics, sunscreens, skin whiteners, antiacne agents, antibacterial agents, antifungal agents, botanical extracts, pharmaceuticals, minerals, plant extracts, concentrates of plant extracts, emollients, moisturizers, skin protectants, humectants, silicones, skin soothing ingredients, colorants, perfumes, and the like. The quantities of such active agents present in the polymeric microparticles are sufficient to perform their intended function, without adversely affecting the benefits of other ingredients present in the wipe product.

More particularly, such an active agent can be one of, or a mixture of, a cosmetic compound, a medicinally active compound, a compound used in cosmetics or personal care, or any other compound that is useful upon topical application to an animate or inanimate surface. Such topically active agents include, but are not limited to, skin-care compounds, plant extracts, antioxidants, insect repellants, counterirritants, vitamins, steroids, antibacterial compounds, antifungal compounds, antiinflammatory compounds, topical anesthetics, sunscreens, optical brighteners, and other cosmetic and medicinal topically effective compounds.

For example, a skin conditioner can be the topically applied compound. Skin conditioning agents include, but are not limited to, humectants, such a fructose, glucose, glycerin, propylene glycol, glycereth-26, mannitol, pyrrolidone carboxylic acid, hydrolyzed lecithin, coco-betaine, cysteine hydrochloride, glucamine, sodium gluconate, potassium aspartate, oleyl betaine, thiamine hydrochloride, sodium laureth sulfate, sodium hyaluronate, hydrolyzed proteins, hydrolyzed keratin, amino acids, amine oxides, water-soluble derivatives of vitamins A, E, and D, amino-functional silicones, ethoxylated glycerin, alpha-hydroxy acids and salts thereof, fatty oil derivatives, such as PEG-24 hydrogenated lanolin, beta-hydroxy acids and salts thereof (e.g., glycolic acid, lactic acid, and salicylic acid), and mixtures thereof. Numerous other skin conditioners are listed in the CTFA Cosmetic Ingredient Handbook, First Ed., J. Nikotakis, ed., The Cosmetic, Toiletry and Fragrance Association (1988), (hereafter CTFA Handbook), pages 79-84, incorporated herein by reference.

The skin conditioner also can be a water-insoluble ester having at least 10 carbon atoms, and preferably 10 to about 32 carbon atoms. Suitable esters include those comprising an aliphatic alcohol having about eight to about twenty carbon atoms and an aliphatic or aromatic carboxylic acid including from two to about twelve carbon atoms, or conversely, an aliphatic alcohol having two to about twelve carbon atoms with an aliphatic or aromatic carboxylic acid including about eight to about twenty carbon atoms. The ester is either straight-chained or branched. Suitable esters, therefore, include, for example, but are not limited to:

(a) aliphatic monohydric alcohol esters, including, but not limited to: myristyl propionate, isopropyl isostearate, isopropyl myristate, isopropyl palmitate, cetyl acetate, cetyl propionate, cetyl stearate, isodecyl neopentanoate, cetyl octanoate, isocetyl stearate;

(b) aliphatic di- and tri-esters of polycarboxylic acid, including, but not limited to: diisopropyl adipate, diisostearyl fumarate, dioctyl adipate, and triisostearyl citrate;

(c) aliphatic polyhydric alcohol esters, including, but not limited to: propylene glycol dipelargonate;

(d) aliphatic esters of aromatic acids, including, but not limited to: C₁₂-C₁₅ alcohol esters of benzoic acid, octyl salicylate, sucrose benzoate, and dioctyl phthalate.

Numerous other esters are listed in the CTFA Handbook, at pages 24 through 26, incorporated herein by reference.

The topically applied compound also can be retinoic acid or a retinol derivative.

The topically applied compound further can be an antioxidant or an optical brightener, like a distyrylbiphenyl derivative, stilbene or a stilbene derivative, a pyralozine derivative, or a coumarin derivative. Optical brighteners useful as the topically applied compound can be any compound capable of absorbing an invisible UV portion of the daylight spectrum, and converting this energy into the longer visible wavelength portion of the spectrum. The optical brightener is colorless on the substrate, and does not absorb energy in the visible part of the spectrum. The optical brightener typically is a derivative of stilbene or 4,4′-diaminostilbene, biphenyl, a 5-membered heterocycle, e.g., triazole, oxazole, or imidazole, or a 6-membered heterocycle, e.g., a coumarin, a naphthalamide, or an s-triazine.

The optical brighteners are available under a variety of tradenames, such as TINOPAL®, LEUCOPHOR®, and CALCOFLUOR®. Specific fluorescent compounds include, but are not limited to, TINOPAL® 5BM, CALCOFLUOR® CG, and LEUCOPHOR® BSB.

In addition, other compounds can be included in a present composition as the topically active compound in an amount sufficient to perform their intended function. For example, sunscreen compounds such as benzophenone-3, tannic acid, uric acids, quinine salts, dihydroxy naphtholic acid, an anthranilate, p-aminobenzoic acid, phenylbenzimidazole sulfonic acid, PEG-25, or p-aminobenzoic acid can be used as the topically applied compound. Further, sunscreen compounds such as dioxybenzone, ethyl 4-[bis(hydroxypropyl)]aminobenzoate, glyceryl aminobenzoate, homosalate, methyl anthranilate, octocrylene, octyl methoxycinnamate, octyl salicylate, oxybenzone, padimate O, red petrolatum, titanium dioxide, 4-menthylbenzylidene camphor, benzophenone-1, benzophenone-2, benzophenone-6, benzophenone-12, isopropyl dibenzoyl methane, butyl methoxydibenzoylmethane, zotocrylene, or zinc oxide can be used as the topically applied compound. Other sunscreen compounds are listed in CTFA Handbook, pages 86 and 87, incorporated herein by reference.

Similarly, topically applied drugs, like antifungal compounds, antibacterial compounds, antiinflammatory compounds, topical anesthetics, skin rash, skin disease, and dermatitis medications, and antiitch and irritation-reducing compounds can be used as the active agent in the compositions of the present invention. For example, analgesics such as benzocaine, dyclonine hydrochloride, aloe vera, and the like; anesthetics such as butamben picrate, lidocaine hydrochloride, xylocalne, and the like; antibacterials and antiseptics, such as povidoneiodine, polymyxin b sulfate-bacitracin, zinc-neomycin sulfate-hydrocortisone, chloramphenicol, ethylbenzethonium chloride, erythromycin, and the like; antiparasitics, such as lindane; essentially all dermatologicals, like acne preparations, such as benzoyl peroxide, erythromycin benzoyl peroxide, clindamycin phosphate, 5,7-dichloro-8-hydroxyquinoline, and the like; antiinflammatory agents, such as alclometasone dipropionate, betamethasone valerate, and the like; burn relief ointments, such as o-amino-p-toluenesulfonamide monoacetate, and the like; depigmenting agents, such as monobenzone; dermatitis relief agents, such as the active steroid amcinonide, diflorasone diacetate, hydrocortisone, and the like; diaper rash relief agents, such as methylbenzethonium chloride, and the like; emollients and moisturizers, such as mineral oil, PEG-4 dilaurate, lanolin oil, petrolatum, mineral wax, and the like; fungicides, such as butocouazole nitrate, haloprogin, clotrimazole, and the like; herpes treatment drugs, such as O-[(2-hydroxymethyl)-methyl]guanine; pruritic medications, such as alclometasone dipropionate, betamethasone valerate, isopropyl myristate MSD, and the like; psoriasis, seborrhea, and scabicide agents, such as anthralin, methoxsalen, coal tar, and the like; steroids, such as 2-(acetyloxy)-9-fluoro-1′,2′,3′,4′-tetrahydro-11-hydroxypregna-1,4-dieno-[16,17-b]naphthalene-3,20-dione and 21-chloro-9-fluoro-1′,2′,3′,4′-tetrahydro-11b-hydroxypregna-1,4-dieno-[16,17-b]naphthalene-3,20-dione. Any other medication capable of topical administration, like skin protectants, such as allantoin, and antiacne agents, such as salicylic acid, also can be incorporated in a composition of the present invention in an amount sufficient to perform its intended function. Other topically applied compounds are listed in Remington's Pharmaceutical Sciences, 17th Ed., Mack Publishing Co., Easton, Pa. (1985), pages 773-791 and pages 1054-1058 (hereinafter Remington's), incorporated herein by reference.

The topically-active agent also can be a plant extract on a natural oil. Nonlimiting plant extracts are those obtained from alfalfa, aloe vera, amla fruit, angelica root, anise seed, apple, apricot, artichoke leaf, asparagus root, banana, barberry, barley sprout, bee pollen, beet leaf, bilberry fruit, birch leaf, bitter melon, black currant leaf, black pepper, black walnut, blueberry, blackberry, burdock, carrot, cayenne, celery seed, cherry, chickwood, cola nut, corn silk, cranberry, dandelion root, elderberry, eucalyptus leaf, flax oil powder, ginger root, gingko leaf, ginseng, goldenrod, goldenseal, grape, grapefruit, guava, hibiscus, juniper, kiwi, kudzu, lemon, licorice root, lime, malt, marigold, myrrh, olive leaf, orange fruit, orange peel, oregano, papaya fruit, papaya leaf, passion fruit, peach, pear, pine bark, plum, pomegranate, prune, raspberry, rice bran, rhubarb root, rosemary leaf, sage leaf, spearmint leaf, St. John's wart, strawberry, sweet cloves, tangerine, violet herb, watercress, watermelon, willow bark, wintergreen leaf, witch hazel bark, yohimbe, and yucca root.

The active agent also can be a deodorant or antiperspirant compound, such as an astringent salt or a bioactive compound. The astringent salts include organic and inorganic salts of aluminum zirconium, zinc, and mixtures thereof. The anion of the astringent salt can be, for example, sulfate, chloride, chlorohydroxide, alum, formate, lactate, benzyl sulfonate, or phenyl sulfonate. Exemplary classes of antiperspirant astringent salts include aluminum halides, aluminum hydroxyhalides, zirconyl oxyhalides, zirconyl hydroxyhalides, and mixtures thereof.

Exemplary aluminum salts include aluminum chloride and the aluminum hydroxyhalides having the general formula Al₂(OH)ₓQ_{y}.XH₂O, wherein Q is chlorine, bromine, or iodine; x is about 2 to about 5; x+y is about 6, wherein x and y are not necessarily integers; and X is about 1 to about 6. Exemplary zirconium compounds include zirconium oxy salts and zirconium hydroxy salts also referred to as zirconyl salts and zirconyl hydroxy salts, and represented by the general empirical formula ZrO(OH)_{2-nz}L₂, wherein z varies from about 0.9 to about 2 and is not necessarily an integer; n is the valence of L; 2-nz is greater than or equal to 0; and L is selected from the group consisting of halides, nitrate, sulfamate, sulfate, and mixtures thereof.

Exemplary deodorant compounds include, but are not limited to, aluminum bromohydrate, potassium alum, sodium aluminum chlorohydroxy lactate, aluminum sulfate, aluminum chlorohydrate, aluminum-zirconium tetrachlorohydrate, an aluminum-zirconium polychlorohydrate complexed with glycine, aluminum-zirconium trichlorohydrate, aluminum-zirconium octachlorohydrate, aluminum sesquichlorohydrate, aluminum sesquichlorohydrex PG, aluminum chlorohydrex PEG, aluminum zirconium octachlorohydrex glycine complex, aluminum zirconium pentachlorohydrex glycine complex, aluminum zirconium trichlorohydrex glycine complex, aluminum chlorohydrex PG, zirconium chlorohydrate, aluminum dichlorohydrate, aluminum dichlorohydrex PEG, aluminum dichlorohydrex PG, aluminum sesquichlorohydrex PG, aluminum chloride, aluminum zirconium pentachlorohydrate, chlorophylli copper complex, numerous other useful antiperspirant compounds known in the art, and mixtures thereof. The active agent also can be a fragrance that acts as a deodorizer by masking malodors.

The polymeric microparticles also can contain cleansing agents for animate and inanimate surfaces, such as an alcohol or a surfactant. The surfactant can be an anionic surfactant, a cationic surfactant, a nonionic surfactant, or a compatible mixture of surfactants. The surfactant also can be an ampholytic or amphoteric surfactant, which have anionic or cationic properties depending on the pH of the composition.

Examples of anionic surfactants include, without limitation, soaps, alkyl sulfates, anionic acyl sarcosinates, methyl acyl taurates, N-acyl glutamates, acyl isethionates, alkyl phosphate esters, ethoxylated alkyl phosphate esters, alkyl sulfosuccinates, trideceth sulfates, protein condensates, mixtures of ethoxylated alkyl sulfates, and the like. Examples of anionic nonsoap surfactants include, without limitation, the alkali metal salts of an organic sulfate having an alkyl radical containing about 8 to about 22 carbon atoms and a sulfonic acid or sulfuric acid ester radical.

Examples of zwitterionic surfactants include, without limitation, derivatives of aliphatic quaternary ammonium, phosphonium, and sulfonium compounds, in which the aliphatic radicals can be straight chain or branched and wherein one of the aliphatic substituents contains an anionic water-solubilizing group, e.g., carboxyl, sulfonate, sulfate, phosphate, or phosphonate. Examples of amphoteric surfactants include, without limitation, derivatives of aliphatic secondary and tertiary amines in which the aliphatic radical can be straight chain or branched and wherein one of the aliphatic substituents contains about 8 to about 18 carbon atoms and one contains an anionic water solubilizing group, e.g., carboxyl, sulfonate, sulfate, phosphate, or phosphonate.

Examples of cationic surfactants include, without limitation, stearyldimethylbenzyl ammonium chloride; dodecyltrimethyl ammonium chloride; nonylbenzylethyldimethyl ammonium nitrate; and tetradecylpyridinium bromide.

Nonionic surfactants include, without limitation, compounds produced by the condensation or ethylene oxide groups with an organic hydrophobic compound, which may be aliphatic or alkyl aromatic in nature, for example, the polyethylene oxide condensates of alkyl phenols.

A present article also can contain water or an organic solvent in the substrate and/or the polymeric microparticles. The solvent can be a water-soluble organic compound containing one to six, and typically one to three, hydroxyl groups, e.g., alcohols, diols, triols, and polyols. Specific examples of solvents include, but are not limited to, methanol, ethanol, isopropyl alcohol, n-butanol, n-propyl alcohol, ethylene glycol, propylene glycol, glycerol, diethylene glycol, dipropylene glycol, tripropylene glycol, hexylene glycol, butylene glycol, 1,2,6-hexanetriol, sorbitol, PEG-4, 1,5-pentanediol, similar hydroxyl-containing compounds, and mixtures thereof. The solvent also can be water or an aprotic solvent, e.g., dimethyl sulfoxide or tetrahydrofuran. A present wipe product also can contain oil, for example, mineral oil or a silicone oil.

Other ingredients also can be incorporated into the substrate and/or the polymeric microparticles to provide a present article of manufacture. These ingredients include, but are not limited to, dyes, fragrances, preservatives, antioxidants, and similar types of compounds. These ingredients are included in an amount sufficient to perform their intended function, without adversely affecting the efficacy of an active agent present in the polymeric microparticles, the polymeric microparticles, or substrate perform its intended function.

The articles of manufacture of the present invention have several practical end uses, including hand cleansers, surgical scrubs, body splashes, antiseptics, disinfectants, hand sanitizer gels, deodorants, and similar personal care products. The articles further can be used on inanimate surfaces, for example, sinks and countertops in hospitals, cruise ships, nursing homes, food service areas, and meat processing plants.

For example, the article of manufacture can be a wipe product for delivery of a nonlimiting active agent disclosed above. The article also can be a pillow case, a bed sheet, or a diaper sheet to impart a benefit to skin contacted by the article. The article can be in the form of an applicator to deliver a desired compound to a contacted surface. The article also can be a self-deodorizing garment, a cloth towel, a tablecloth, a paper towel, or a napkin.

The present articles can be designed as cosmetic basecoats and undercoats, blushers, face, body, and hand creams and lotions, cosmetic foundations, hormone creams and lotions, leg and body paints, makeup bases, makeup fixatives, makeup products, moisturizing creams and lotions, night creams and lotions, paste masks, skin care products, skin fresheners, skin lighteners, tonics, dressings, and wrinkle smoothing creams and lotions.

In particular embodiments, the articles can be designed as lotions; makeup preparations, like makeup foundations; skin care preparations, like hand lotions, vanishing creams, night creams, sunscreens, body lotions, facial creams, clay masks, moisturizing lotions, makeup removers, antiacne preparations, antiaging preparations, and sebum control preparations; analgesic and cortisonal steroid creams and preparations; insect repellants; and facial masks and revitalizers.

The polymeric microparticle delivery system can be applied to any surface of the substrate that is desired to be the working surface of the article. The articles can have more than one working surface, i.e., both external surfaces of the substrate, or the substrate be treated with the polymeric microparticle delivery system in a pattern, or only on a portion of the available substrate surface area.

An article of the present invention is topically applied to a target surface, such as skin, as needed in order to impart a desired benefit. The preferred method of use is rubbing the substrate working layer onto a target surface with a soft massage to ensure intimate contact with the target surface.

As discussed above, both animate and inanimate surfaces can be treated with an article of the present invention. A particularly important surface is mammalian skin, and particularly human skin. However, the present method also is useful in treating inanimate surfaces of all types.

The present articles are useful to treat hard surfaces. As used herein with respect to the surfaces treated by the present articles, the term “hard” refers to surfaces comprising refractory materials, such as glazed and unglazed tile, brick, porcelain, ceramics, metals, glass, and the like, and also includes wood and hard plastics such as formica, polystyrenes, vinyls, acrylics, polyesters, and the like. Such surfaces are found, for example, in kitchens and bathrooms. A hard surface can be porous or nonporous.

A present article also can be used to treat hard surfaces in processing facilities (such as dairy, brewing, and food processing facilities), healthcare facilities (such as hospitals, clinics, surgical centers, dental offices, and laboratories), long-term healthcare facilities (such as nursing homes), farms, cruise ships, schools, and private homes.

A present article can be used to treat environmental surfaces such as floors, walls, ceilings, and drains. The article can be used to treat equipment such as food processing equipment, dairy processing equipment, brewery equipment, and the like. The article can be used to treat a variety of surfaces including food contact surfaces in food, dairy, and brewing facilities, countertops, furniture, sinks, and the like. The article further can be used to treat tools and instruments, such as medical tools and instruments, dental tools and instruments, as well as equipment used in the healthcare industries and institutional kitchens, including knives, wares (such as pots, pans, and dishes, cutting equipment, and the like. Methods of treating hard surfaces are described in U.S. Pat. Nos. 5,200,189; 5,314,687; and 5,718,910, the disclosures of which are incorporated herein by reference in their entirety.

The article can be a textile, such as clothing, protective clothing, laboratory clothing, surgical clothing, patient clothing, carpets, bedding, towels, linens, and the like.

Treatable inanimate surfaces include, but are not limited to, exposed environmental surfaces, such as tables, floors, walls; kitchenwares, including pots, pans, knives, forks, spoons, and plates; food cooking and preparation surfaces, including dishes; food preparation equipment; and tanks, vats, lines, pumps, hoses, and other process equipment. One useful application of the article is to contact dairy processing equipment, which is commonly made from glass or stainless steel. Such equipment can be found both in dairy farm installations and in dairy plant installations for the processing of milk, cheese, ice cream, and other dairy products.

An article of the present invention also can be used in the manufacture of beverages, including fruit juice, dairy products, malt beverages, bottled water products, teas, and soft drinks.

The article of the present invention also can be used to treat medical carts, medical cages, and other medical instruments, devices, and equipment. Examples of medical apparatus treatable by the present method are disclosed in U.S. Pat. No. 6,632,291, incorporated herein by reference.

The present invention, therefore, encompasses swiping a wipe product of the present invention over inanimate surfaces, such as household surfaces, e.g., countertops, kitchen surfaces, food preparing surfaces (cutting boards, dishes, pots and pans, and the like); major household appliances, e.g., refrigerators, freezers, washing machines, automatic dryers, ovens, microwave ovens, and dishwashers; cabinets; walls; floors; bathroom surfaces, shower curtains, garbage cans, and/or recycling bins, and the like.

To demonstrate the new and unexpected benefits provided by an article of the present invention, the following tests were performed.

### Test Procedures

### Determination of Particle Transfer to Dark Skin via Skin Whitening

### Instrument:

SP 62S Sphere Spectrophotometer, available from X-Rite Corporation, Grandville, Mich. 49418.

### Procedure:

Wash skin and define test area.

Measure test area color with the SP 62S Sphere Spectrophotometer prior to application of the wipe using CIELAB (L*a*b*) measurement technique (baseline).

Apply a wipe product to the skin test area in controlled manner.

Allow the treated skin to dry.

Measure the treated skin color as above.

### Calculation:

The absolute color change ΔE* for the treated skin is calculated using the CIELAB color system by the equation:

ΔE*ab=[(ΔL²)+(Δa²)+(Δb2)]^{1/2}

The magnitude of the ΔE*ab value is a measure of the whitening of skin, and indicates the relative amount of skin whitening compared to skin whitening obtained by applying a lotion containing the same active ingredients, in the same amounts, directly to the skin.

### FTIR Analysis for Dimethicone on Human Skin

The test procedure was adapted from a method provided by the Dow Corning Corporation:

### Instrument

Perkin Elmer Spectrum One FTIR spectrophotometer; HATR Sampling Assembly; ZnSe Flat Plate 45°.

### Procedure:

Wash forearm with silicone-free soap and define a test area of forearm skin.

Measure FTIR spectrum of the untreated forearm test area (baseline).

Apply wipe by gently rubbing the working layer of the wipe on target area five times, repositioning the wipe, rubbing five more times, again repositioning the wipe, and again rubbing five times (for a total of 15 rubs).

Allow the forearm to dry for 30 minutes.

Measure FTIR spectrum of the treated test areas of the forearm.

### Calculation:

The relative concentrations of dimethicone on the skin were determined from the ratio of the area of the dimethicone (1260 cm⁻¹) peak to the area of the skin amide peak (1545 cm⁻¹). This ratio then is corrected by subtracting the ratio obtained from the same peak locations on the skin baseline FTIR. The results were reported as dimethicone peak intensity, with larger numbers indicating a greater transfer of dimethicone to the skin.

### Particle Size Determination

Particle size data for the polymeric microparticles was provided by the particle manufacturer.

Table I contains nonlimiting examples of substrates suitable for use in the present invention. The porosity of the substrates is indicated by the corresponding Frazier numbers.

TABLE 1
Substrates

| | Frazier | | | | |
|---|---|---|---|---|---|
| Nonwoven | Air | | Thickness | | |
| Product | (Lpm)¹⁾ | GSM²⁾ | Microns | Description | supplier |
| A | 34.47 | 38 | 250 | Hydroentangled | Ahlstrom³⁾ |
| | | | | natural and | |
| | | | | synthetic fibers; | |
| | | | | latex binder free | |
| B | 62.52 | 54 | 450 | Cellulose and | Ahlstrom |
| | | | | synthetic; | |
| | | | | latex free | |
| C | 163.48 | 55 | 550 | 70% viscose, | Ahlstrom |
| | | | | 30% PET | |

¹⁾Lpm is liters per minute
²⁾GSM is grams per square meter
³⁾Ahlstrom Windsor Locks LLC, Windsor Locks, CT.

Table II contains nonlimiting examples of polymeric microparticle delivery systems suitable for use in the present invention.

TABLE II
Microparticle Delivery Systems

| | PSD | |
|---|---|---|
| Microparticles | (microns) | Supplier |
| POLYTRAP 6603 | 60 | AMCOL Health & Beauty |
| | | Solutions, Inc. |
| POLY-PORE © E200 | 30 | AMCOL Health & Beauty |
| | | Solutions, Inc. |
| MICROSPONGE CO80A | 20 | AMCOL Health & Beauty |
| | | Solutions, Inc. |
| DC 9506 | 4 | Dow Corning |

¹⁾PSD is median particle size diameter.

### EXAMPLE 1

### Transfer of Particulate Delivery Systems to a Target Surface (Skin)

To determine the ability of a present wipe product to deliver polymeric microparticles to the skin, several lotions were prepared containing the polymeric microparticle delivery systems listed in Table II. The lotions were designed as simple systems that allow the detection of particles on the target surface (dark human skin). The lotion compositions are summarized in Table III.

TABLE III

| | Sample | | |
|---|---|---|---|
| | 2% POLY- | 2% POLY- | |
| | TRAP 6603 | PORE © E200 | 5% DC9506 |
| Ingredient | Wt % | Wt % | Wt % |
| Deionized Water | 84.74 | 84.74 | 81.74 |
| Carbopol Ultrez 21 | 0.25 | 0.25 | 0.25 |
| EDTA | 0.01 | 0.01 | 0.01 |
| Alcohol | 12.00 | 12.00 | 12.00 |
| Phenonip (preservative) | 1.00 | 1.00 | 1.00 |
| NaOH (50%) | to pH 6.5 | to pH 6.5 | to pH 6.5 |
| POLYTRAP 6603 | 2 | — | — |
| POLY-PORE © E200 | | 2 | |
| DC9506 | — | — | 5 |

The lotions were applied to a working layer of a 3×3-inch square wipe substrate having a Frazier Porosity number of 62.5 L/min/100 cm² (12.7 mm water) at a loading of 1 gram/wipe. The substrates then were applied to dark human skin (as described in the test method section), allowed to dry, and measured for color change, as described in the test procedure section above. When the lotion dries on the skin, the particles are observed as a whitening of the skin, and the measurement of the absolute color change gives a measure of the efficiency of transfer of the particles to the skin. The results were compared to a lotion applied directly to the skin, which provides a measure of skin whitening that occurs for 100% transfer of the particles to the skin.

The results of the test are summarized in Table IV. The results show, in accordance with the invention, for a given Frazier porosity, % Particle Transfer to the target surface increases with increasing mean particle size diameter.

TABLE IV
Particle Transfer Data (Frazier No. 62.5)

| | | | % Particle |
|---|---|---|---|
| | | | Transfer |
| | Delta E Wipe | Delta E Lotion | vs. Lotion |
| 2% POLYTRAP 6603 | 5.71 | 6.32 | 90.4 |
| 2% POLY-PORE © E200 | 3.30 | 8.33 | 39.6 |
| 5% DC9506 | 0.92 | 3.29 | 27.9 |

### EXAMPLE 2

### Transfer of Active to a Target Surface (Skin)

The effect of substrate Frazier Number on transfer of polymeric microparticles to a target surface was demonstrated using a microparticle delivery system loaded with dimethicone, a skin protecting active agent. The preparation of the polymeric microparticle delivery system containing dimethicone and corresponding lotion were prepared as follows:

POLYTRAP 6603 (3.75 grams) was added to a glass beaker. Dimethicone (Dow Corning DC100-350CS; 15.00 grams) was added slowly to the beaker while the POLYTRAP 6603 powder was agitated with a glass stirring rod. Agitation was continued until the dimethicone had been adsorbed by the POLYTRAP 6603 yielding 18.75 grams of a free-flowing, dense powder.

The dimethicone-containing POLYTRAP 6603 then was used in the formulation of a simple lotion as shown in Table V.

TABLE V

| Ingredient | Grams |
|---|---|
| Deionized Water | 485 |
| Carbopol Ultrez 10 | 1.25 |
| Tetrasodium EDTA | 0.56 |
| NaOH | to pH = 6.5 |
| POLYTRAP-Dimethicone | 18.75 |
| Microparticles | |

The lotion of Table V was a viscous suspension of the particulate delivery system having 2.97%, by weight, dimethicone contained in the POLYTRAP particles. The lotion of Table V then was used to create lotion/substrate combinations as follows:

Substrates of different Frazier numbers were cut into 3-inch squares. The working surface of each square was coated with one gram of the lotion. A summary of the lotion substrate combinations is given in Table VI.

TABLE VI

| | | Frazier Air | Lotion Loading |
|---|---|---|---|
| Sample | Substrate | (Lpm) | (grams) |
| 1 | Ahlstrom 10378 | 34.47 | 1 |
| 2 | Ahlstrom 18017 | 62.52 | 1 |
| 3 | Ahlstrom 17006 | 163.48 | 1 |

The wipes then were applied to human skin as described in the test procedures section. The skin was allowed to dry, then analyzed by FTIR. The results (Table VII) show, in accordance with the invention, that the reduction in Frazier number improves the transfer of polymeric microparticles to the skin.

TABLE VII
Dimethicone Transfer to Skin: POLYTRAP Lotion

| Frazier Number (Lpm) | Dimethicone Peak Intensity |
|---|---|
| 34.5 | 93.3 |
| 62.5 | 71.2 |
| 163.5 | 42.0 |

### EXAMPLE 3

### Advantages of Invention Over Conventional Technology

As an example of the invention, a wipe composition of the invention was compared to a commercially available wipe product that contained dimethicone in a conventional emulsion system for the transfer of dimethicone to the skin.

The present wipe was described in Table VI, Sample 2. The commercially available wipe product was the Sage Comfort Shield perineal care washcloth. The commercial product is an over-the-counter skin protection wipe product containing 3% dimethicone content, as listed on its drug facts label. The Sage wipe was cut to a 3-inch square size to have an equal surface area to the wipe composition of the invention.

The wipe products were compared using the FTIR method described in the test section, with the exception that the wipes were applied with 9 total rubs rather 15. A second FTIR analysis was performed 60 minutes after the wipe application, and after a one minute warm water rinse of the test area. The test results (Table VIII) are the average of test results for four subjects.

TABLE VIII

Dimethicone Peak Intensity

| | 30 minutes after | |
|---|---|---|
| | application | After rinse |
| Present Wipe Product | 33.18 | 10.8 |
| Sage Products Commercial | 0.87 | 0.46 |
| Wipe | | |

The results show that a wipe composition of the invention outperforms the commercial wipe product in an ability to transfer the active agent i.e., dimethicone, to the target surface, i.e., human skin. This performance advantage remains after a rinse demonstrating that an inventive composition outperforms a commercial product in imparting long-lasting skin protection.

Obviously, many modifications and variations of the invention as hereinbefore set forth can be made without department from the spirit and scope thereof, and, therefore, only such limitations should be imposed as are indicated by the appended claims.

What is claimed is:

1. An article of manufacture comprising a porous substrate having a Frazier number of less than 210 and having polymeric microparticles of median particle size diameter greater than 1 micron impregnated in the porous substrate.

2. The article of claim 1 having a Frazier number of about 30 to less than 210.

3. The article of claim 2 having a Frazier number of less than 185.

4. The article of claim 2 having a Frazier number of less than 100.

5. The article of claim 1 wherein the porous substrate comprises a fiber, fiber blends, a cellulosic fibrous web, a woven web, a nonwoven web, a spun-lace fabric, a melt-blown fabric, an air-laid fabric, a paper fabric, a polymeric film, a polymeric foam, a perforated film, and mixtures thereof.

6. The article of claim 1 wherein the porous substrate comprises a single sheet, pad, or applicator.

7. The article of claim 1 wherein the porous substrate comprises a plurality of laminated sheets and/or pads.

8. The article of claim 1 wherein the polymeric microparticles have a mean particle size diameter of greater than 1 micron to about 3000 microns.

9. The article of claim 1 wherein the polymeric microparticles are present in an amount of about 0.5% to about 40%, by weight, of the wipe product.

10. The article of claim 1 wherein the polymeric microparticles are highly crosslinked and are derived from methacrylate monomers, acrylate monomers, or mixtures thereof.

11. The article of claim 1 wherein the polymeric microparticles comprise an allyl methacrylate copolymer, an ethylene glycol dimethacrylate/allyl methacrylate copolymer, a lauryl methacrylate/ethylene glycol dimethacrylate copolymer, and mixtures thereof.

12. The article of claim 1 wherein the polymeric microparticles are selected from the group consisting of a copolymer of allyl methacrylate and ethylene glycol dimethacrylate, a copolymer of ethylene glycol dimethacrylate and lauryl methacrylate, a copolymer of methyl methacrylate and ethylene glycol dimethacrylate, a copolymer of 2-ethylhexyl acrylate, styrene, and divinylbenzene, and mixtures thereof.

13. The article of claim 1 wherein the polymeric microparticles comprise a copolymer of allyl methacrylate and ethylene glycol dimethacrylate, a copolymer of ethylene glycol dimethacrylate and lauryl methacrylate, or a mixture thereof.

14. The article of claim 13 wherein the polymeric microparticles comprise copolymer of ethylene glycol dimethacrylate and lauryl methacrylate.

15. The article of claim 1 wherein the polymeric microparticles further comprise a loading of about 0.01% to about 80% of an active agent, by weight, of the loaded microparticles.

16. The article of claim 15 wherein the active agent is present in an amount of about 1% to about 70%, by weight, of the loaded microparticles.

17. The article of claim 16 wherein the active agent is present in an amount of about 1% to about 1% to about 50%, by weight, of the loaded microparticles.

18. The article of claim 15 wherein the active agent comprises a compound selected from the group consisting of a pesticide, a drug, a therapeutic agent, a deodorant, a hormone, an analgesic, an antiacne agent, a pharmaceutical, a skin conditioner, a skin protectant, a skin-soothing agent, an antioxidant, an insect repellant, a counterirritant, a vitamin, a plant extract, a steroid, an antibacterial compound, an antifungal compound, an antiinflammatory compound, a topical anesthetic, an epidermal lipid replacement, an antiaging compound, a sunscreen, a skin whitener, an optical brightener, an antiperspirant, a disinfectant, a sanitizer, a dermatitis or skin disease medication, a cleanser, an alcohol, and mixtures thereof.

19. The article of claim 18 wherein the active agent is selected from the group consisting of benzocaine, dyclonine hydrochloride, aloe vera, butamben picrate, lidocaine hydrochloride, xylocaine, providone-iodine, polymyxin b sulfate-bacitracin, zinc-neomycin sulfate-hydrocortisone, chloramphenicol, ethylbenzethonium chloride, erythromycin, lindane, benzoyl peroxide, erythromycin benzoyl peroxide, clindamycin phosphate, 5,7-dichloro-8-hydroxyquinoline, alclometasone dipropionate, betamethasone valerate, o-amino-p-toluenesulfonamide monoacetate, monobenzone, amcinonide, diflorasone diacetate, hydrocortisone, methylbenzethonium chloride, PEG-4 dilaurate, lanolin oil, petrolatum, mineral wax, butocouazole nitrate, haloprogin, clotrimazole, O-[(2-hydroxymethyl)methyl]guanine, alclometasone dipropionate, betamethasone valerate, isopropyl myristate MSD, anthralin, methoxsalen, coal tar, 2-(acetyloxy)-9-fluoro-1′,2′,3′,4′-tetrahydro-1-hydroxypregna-1,4-dieno-[16,17-b]naphthalene-3,20-dione, 21-chloro-9-fluoro-1′,2′,3′,4′-tetrahydro-11b-hydroxypregna-1,4-dieno-[16,17-b]naphthalene-3,20-dione, allantoin, salicylic acid, isopropyl myristate, ascorbic acid, retinol, zinc pyrithione, benzophenone-3, a fragrance, glycolic acid, hyalauronic acid, hydrogen peroxide, a protein, an enzyme, an astringent, a surfactant, tocopherol, butein, hydroquinone, kojic acid, jojoba oil, an alpha or beta hydroxy acid, and mixtures thereof.

20. The article of claim 1 wherein the article is a skin lotion, a cream, a body rinse, a skin cleansing agent, a skin coloring composition, a foundation liquid, a foundation powder, a topical medicament, or a skin treatment, a hard surface cleanser, or a hard surface disinfectant.

21. The article of claim 15 wherein the loaded microparticles further comprise a barrier layer in an amount of about 10% to about 70%, by total weight of the loaded microparticles.

22. The article of claim 21 wherein the barrier layer comprises a compound selected from the group consisting of a poly(acid), a polyol, a salt of a C₈-C₂₀ fatty acid, an alkanolamide, a water-soluble polymer, a biological polymer, a gum, a carbohydrate, a cellulose derivative, a sorbitan derivative, and mixtures thereof.

23. The article of claim 1 wherein the article is selected from the group consisting of a wipe product, a sheet of a diaper, a bed sheet, a garment, a pillow case, an applicator, a paper towel, a cloth towel, a napkin, a carpet, a tablecloth, patient clothing, protective clothing, surgical clothing, laboratory clothing, linens, and gloves.

24. A method of imparting a benefit to a surface comprising contacting the surface with an article of claim 1.

25. A method of imparting a benefit to a surface comprising contacting the surface with an article of claim 15.

26. The method of claim 24 wherein the surface is an animate surface.

27. The method of claim 26 wherein the animate surface is human skin or human hair.

28. The method of claim 24 wherein the surface is an inanimate surface.
